(19) United States
(12) Patent Application Publication
(10) Pub. No.: US 2012/0312310 A1
(43) Pub. Date: Dec. 13, 2012

(54) Heel Protection Device, Buffer Member and Method

(75) Inventor: Max Paul Rogmans, Heiloo (NL)

(73) Assignee: MAXXCARE B.V., Heiloo (NL)

(21) Appl. No.: 13/383,145

(22) PCT Filed: Jul. 7, 2009
(86) PCT No.: PCT/NL09/50410

§ 371 Date: Mar. 2, 2012

(51) Int. Cl. A61F 13/06; B23P 17/04

(52) U.S. Cl. 128/894; 29/428

(57) ABSTRACT

The present invention relates to a heel protection device for protecting a heel against damage or irritation, such as decubitus symptoms, comprising: an encasing body for encasing a part of the lower leg and/or the foot, wherein the encasing body comprises at least a buffer member holder for holding at least one buffer member, one or more buffer members which can be arranged in the encasing body for the purpose of supporting the heel and/or parts surrounding the heel, wherein: the encasing body can be deformed for close fitting to the heel, and the buffer members comprise at least a fluid chamber which can be sealed by means of a substantially fluid-tight membrane which is deformable.

The present invention relates to a heel protection device for protecting a body to be protected against physical damage or irritation, such as, but not limited to, decubitus symptoms. The present invention also relates to a buffer member. The present invention further relates to a method for manufacturing a heel protection device.

A common problem is that specific tendons, bones, protruding body parts and/or skin and other tissue located therearound are damaged. This damage can for instance occur when lying in one position for a long time or in the case of frequent contact pressure on said parts. In a specific application situation such problems are usually referred to as so-called ‘decubitus’. Common solutions here-for relate to modifying a standard mattress such that the load is reduced. Another common solution is a device with so-called cushioning effect arranged around the body. Both such solutions have only a limited effect and after a certain period of time result in the damage recurring.

It is an object of the present invention to provide a solution to such problems. The present invention therefore provides a heel protection device for protecting a heel against damage or irritation, such as decubitus symptoms, comprising:
an encasing body for encasing a part of the lower leg and/or the foot, wherein the encasing body comprises at least a buffer member holder for holding at least one buffer member,
one or more buffer members which can be arranged in the encasing body for the purpose of supporting the heel and/or parts surrounding the heel, wherein:
the encasing body can be deformed for close fitting to the heel, and
the buffer members comprise at least a fluid chamber which can be sealed by means of a substantially fluid-tight membrane which is deformable.

An advantage of such a heel protection device according to the present invention is that the pressure exerted on the body part to be protected against damage can to a great extent be distributed. A point load is hereby converted into a surface load over a large surface area of the body tissue, whereby the decubitus symptoms can be prevented or can at least be postponed for very long periods of time. The application of a buffer member comprising a fluid chamber provides the option of the pressure being displaced from an easily damaged body part to a more robust body part and/or that pressure can be distributed over a larger part of the tissue of a body. An example of a liquid-containing variant is that the device can be freely applied, independently of ambient air pressure, wherein the expansion of a gas is of less importance.

The pressure is here also spread over this more robust body part in that these more robust body parts share a relatively great large area with the buffer member, for instance due to the deformability thereof. A further important advantage is that a device according to the present invention can be applied at all stages of decubitus in order to alleviate the symptoms, prevent further damage and stimulate healing.

In a first preferred embodiment at least one of the buffer members is arranged in the heel protection device such that at least a part thereof supports the foot and/or the lower leg when the lower leg is lying flat on the calf. An advantage hereof is that the load on the heel due to protruding of the heel relative to the lower leg is further prevented. This load is transferred to the lower leg and the heel is held in freely suspended position.

This effect is otherwise achieved by means of an embodiment wherein a buffer member is arranged transversely relative to and opposite the Achilles tendon in the heel protection device. Holding the heel freely suspended is hereby made possible in an alternative manner. A combination of these embodiment aspects provides substantially a sum of the heights which the heel can reach relative to the surface. Such a combination of buffer members therefore makes it possible to achieve the object in highly advantageous manner.

In order to achieve a positive buffer effect for the purpose of protecting the side of the foot, at least one of the buffer members is arranged in a further embodiment such that at least a part thereof supports the foot when it resting on one of the sides thereof. The device hereby also provides a positive protection of the side of the foot.

The gastight membrane preferably comprises a plastic, such as a PE, EVOH, PVDC and the like. An advantage of such a plastic is that it is highly durable and almost absolutely airtight, whereby the buffer member does not need replacing during the lifespan of the heel protection device. It is further possible to clean a heel protection device with a gastight membrane between successive uses thereof. A standard cleaning device for textile can for instance be applied here.

In a further preferred embodiment the buffer member is partially filled with a gas, such as 20-100%, 30-90%, 40-90%, 50-90%, 60-90%, 70-90% and the like. An advantage hereof is that temperature fluctuations can be compensated. A further advantage is that a relatively large contact surface can be obtained between the buffer member with the encasing parts of the heel protection device and the parts of the body to be protected which come into contact therewith. A relatively high air pressure in the buffer member could increase the load on the contact parts of the body for protecting.

The buffer members can preferably be arranged along the body parts for protecting. An example hereof is two buffer members each extending during use at least partially along the Achilles tendon above the ankle. A further example is that at least a part of such buffer members extends during use round the heel of the foot and the Achilles tendon. Such aspects can be envisaged individually or in combination within the concept of the present invention, and provide advantages in the protection of these two body parts. The heel of the foot can for instance be situated suspended between the buffer members extending round the heel so that the protruding part of the heel can be freely suspended while the foot rests on the surrounding part via the buffer members. The normal lying position can here be maintained, while the heel is not loaded.

At least one of the buffer members preferably has an elongate cushion shape. In another embodiment at least one of the buffer members has a substantially point-symmetrical shape in one view, such as round, square, rhombic, polygonal and the like. This latter is for instance particularly suitable for protecting a heel.

In order to provide a lateral support the buffer members can preferably be arranged in a manner such that, when used with a foot, they extend on either side of the foot. A lateral support is herein provided, and protection is provided to the heel and to the Achilles tendon while the leg is situated in the horizontal direction or in a slightly upright direction.

The encasing body more preferably comprises an at least partial sleeve construction. The device can hereby be pulled over the body part for protecting and subsequently fixed.

For the purpose of a fixation the encasing body comprises temporary fastening means such as velcro or laces. It hereby becomes possible to properly modify the device to the shape of the body part for protecting.

In the heel protection device according to a further embodiment the buffer member holder has a form such that it can hold the buffer member or the buffer members close-fittingly in relation to the part of the body to be protected from damage. It becomes possible here to provide a device suitable for long-term use, wherein the protection is precisely defined after manufacture thereof.

A further aspect of the present invention relates to a buffer member according to the present invention for applying in a heel protection device according to the present invention.

A further aspect of the present invention relates to the use of a heel protection device according to the present invention in preventing damage to a body.

A further aspect of the present invention relates to a method for manufacturing a heel protection device according the present invention, wherein the method comprises steps for:
manufacturing an encasing body,
arranging the buffer member or the buffer members in an encasing body.

Further advantages, features and details of the present invention will be described in greater detail hereinbelow on the basis of preferred embodiments which are shown in the accompanying figures. In the figures:

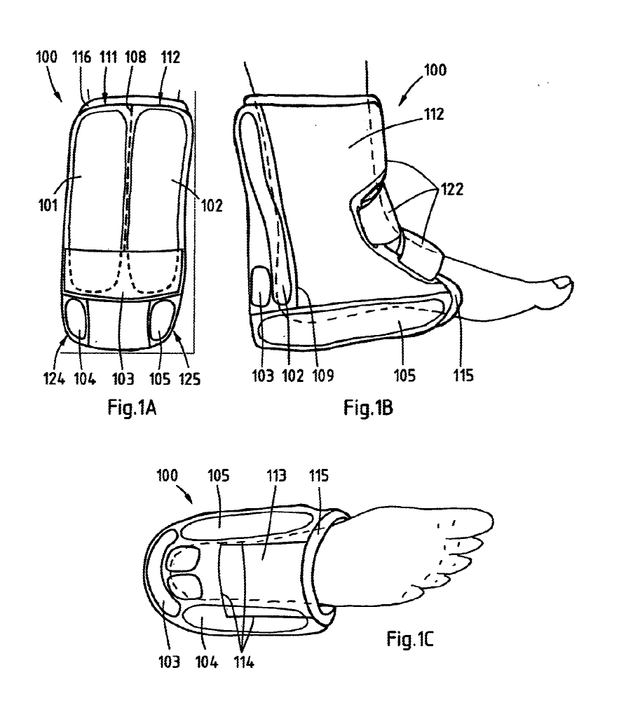

FIG. 1A-1C show a number of views of a first preferred embodiment comprising a fluid chamber according to the present invention.

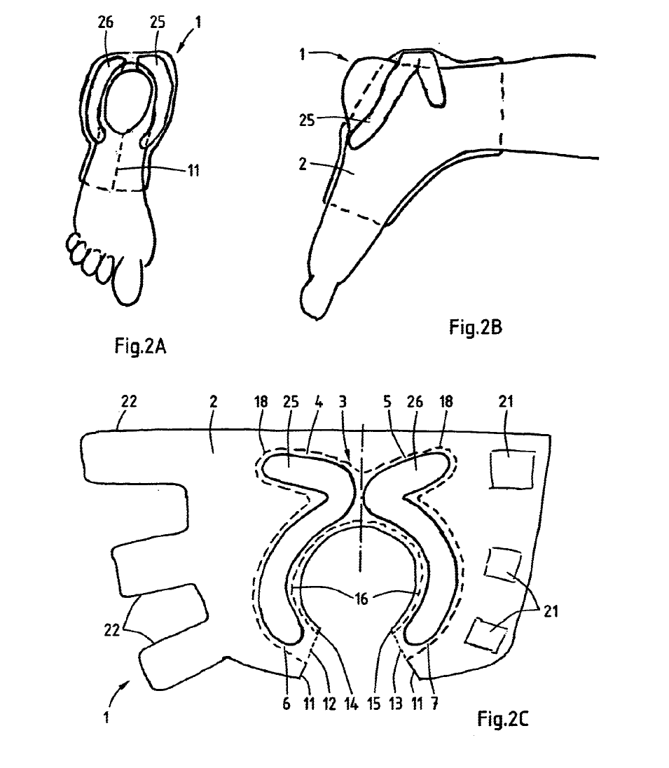

FIG. 2A-2D show a number of views of a first preferred embodiment comprising a fluid chamber according to the present invention.

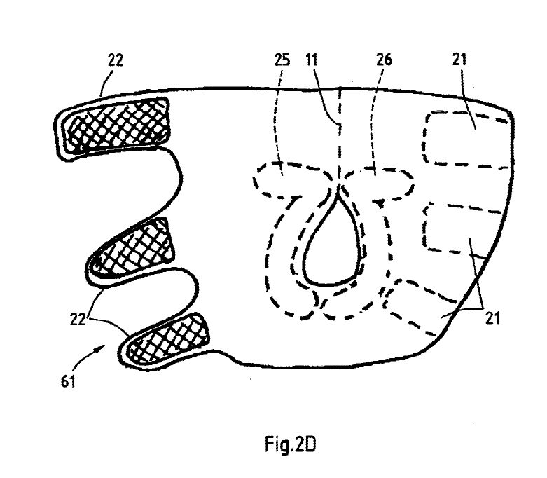

FIG. 3A-3C show a number of views of a further preferred embodiment comprising a fluid chamber according to the present invention.

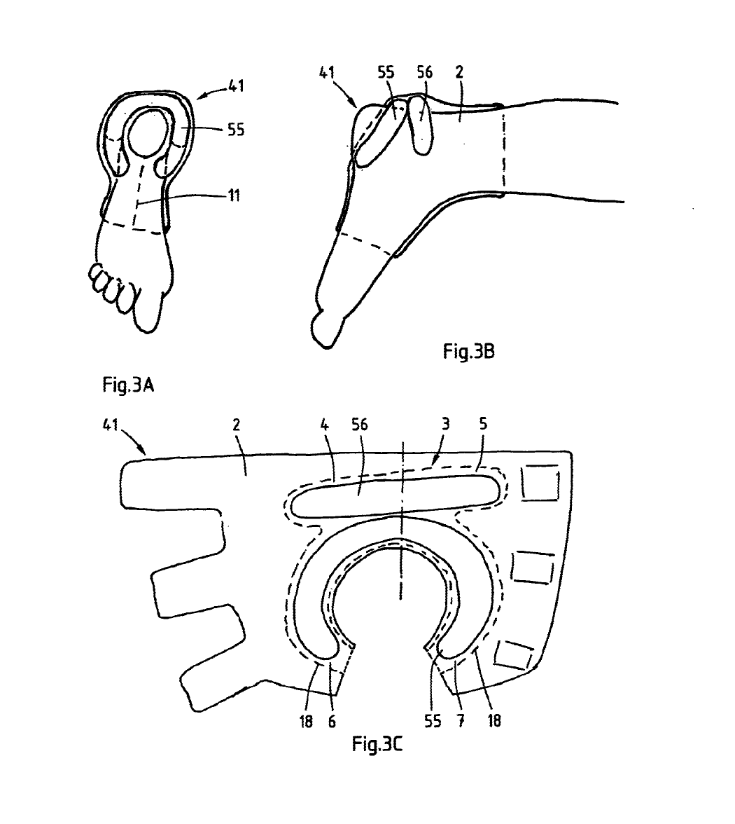

FIG. 4A-4D show a number of views of a further preferred embodiment comprising a fluid chamber according to the present invention.

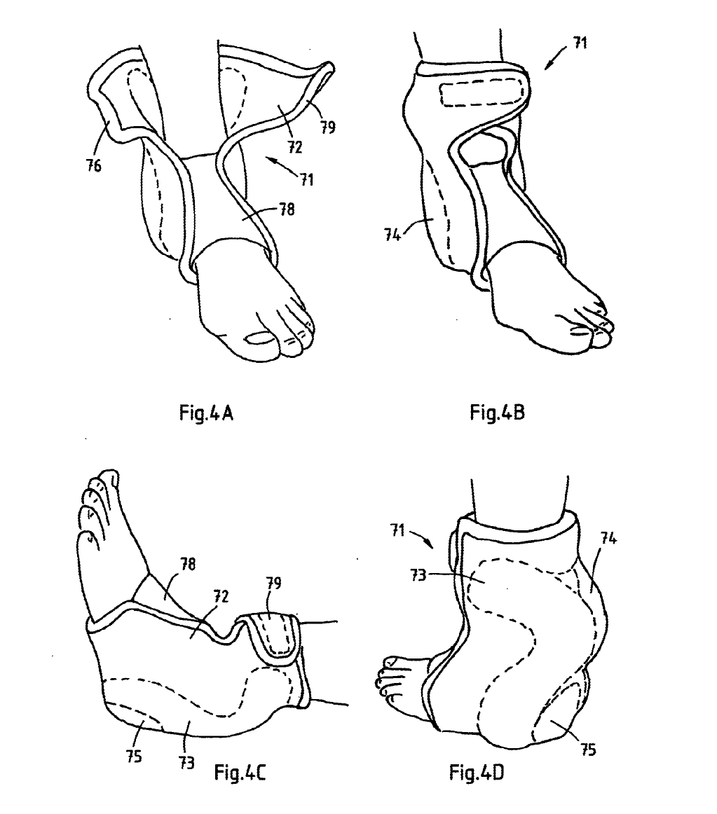

FIG. 5A-5C show a number of views of a further preferred embodiment comprising a fluid chamber according to the present invention.

A first embodiment (FIG. 1) relates to a heel protector 100 for protecting a heel from for instance decubitus or other types of damage which can occur when a person is bedridden, and lying particularly on his/her back.

Heel protector 100 comprises a body 112 enclosing the foot, ankle and lower leg and provided with velcro fasteners 122. By means of the velcro fasteners the body 112 can be arranged round the foot and fastened fitting to the foot. A firm and also comfortable ‘fit’ is hereby obtained. The risk of so-called clubfoot is further also limited in that the foot can be oriented by means of the fastening means such that it is prevented from being held in elongated position for a long time. The consequences hereof are described in greater detail hereinbelow.

On the rear side this heel protector 100 is provided with a dual holder chamber 111, 112. It is a dual holder chamber because chambers 111, 112 are mutually separated by means of a stitched seam 108. Two air cushions 101, 102 can be arranged respectively in the dual holder chamber 111, 112. These air cushions serve to provide a support on substantially both sides of the lower leg and the ankle. This arrangement provides support on either side of the Achilles tendon. The Achilles tendon is hereby spared, while the lower leg and ankle are supported on both sides.

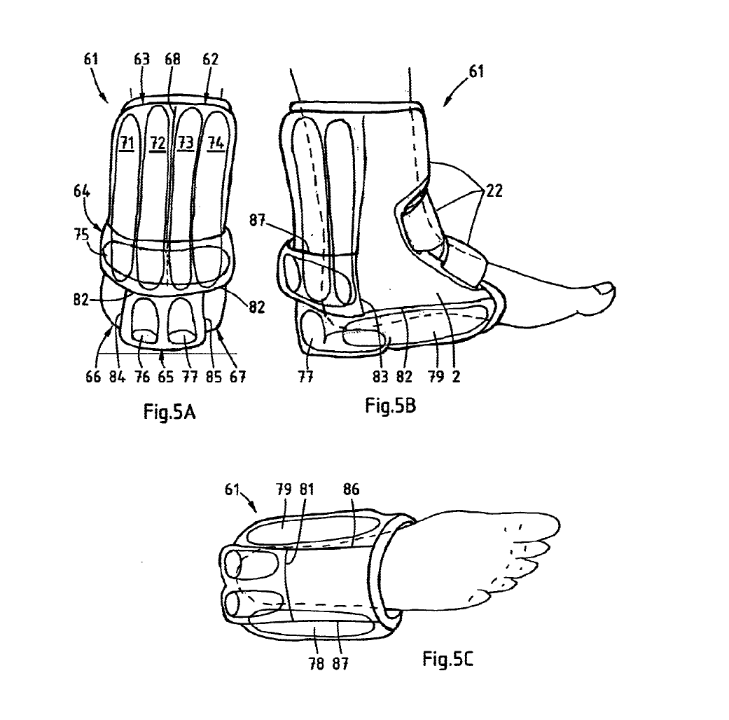

A transversely arranged air cushion 103 is also arranged on the underside of the dual holder chamber 111, 112. In addition to the stated protection of the Achilles tendon, this air cushion provides extra height when the leg is resting thereon whereby, when this heel protector is applied, the heel need not make contact with the surface on which the leg is resting. If there is still a certain measure of residual contact between the heel and the surface, the forces thereof will be minimal. Such a residual contact is for instance possible when the leg rests on a very soft mattress into which the air cushions can sink deeply. Because the air cushions 102 and 101 extend behind air cushion 103 in the view of FIG. 1A, the Achilles tendon continues to be spared even if a transverse cushion such as 103 is applied. This mutual arrangement of the air cushions 102 and 103 is shown particularly well in the side view of FIG. 18.

Two air cushions 104, 105 are arranged on either side of the underside of the heel protection. These air cushions serve to support the foot when it is lying on its side. A protection for the relevant side of the foot is hereby provided on the one hand, and an additional protection for the ankle is provided on the other if the heel protector is situated round the foot when a person is lying on his/her side.

FIG. 1C shows a cut-away bottom view of heel protector 100. On the underside the heel protector is provided with a tread 113 arranged between stitched seams 114. This tread also makes it possible for the heel protector to be worn when walking. This advantageously prevents the heel protector having to be put on or taken off frequently. FIG. 1C further provides a free view of the mutual arrangement of said air cushions.

A further example of wear which occurs is the result of so-called ‘digging’, wherein a person lying on his/her back makes a movement between a stretched leg and a bent leg, wherein the heel makes contact with the sheet and/or the mattress. Such a movement creates a load, comprising a shear force, on the heel which can result in uncomfortable symptoms. This so-called ‘digging’ can occur intentionally or unintentionally and need not be an elongate movement, but can also be an almost stationary ‘wiggling’ of the heel and the lower leg. In the application thereof the heel protector is arranged in the shown manner round the foot and the ankle and the lower leg, or at least parts thereof. The heel protector can here be fastened by means of elongate tongues 22 comprising velcro and velcro strips 21 on the other side of the heel protector.

Different preferred embodiments of the buffer members are described hereinbelow. Both foam bodies and encasing bodies comprising gas-tight spaces can be applied here. The following description is couched in terms of air cushions in which foam bodies can also be applied within the concept of the present invention. Encasing body 2 of the heel protector is manufactured from at least partly multi-layer textile to enable forming of a holder or placing space 6 between the layers for arranging therein air cushions, such as air cushions 25, 26 and air cushions 55, 56 in this embodiment.

Holder space 3 is designed such that air cushions remain in the desired orientation or arrangement and can achieve the positive effect in this arrangement. The space is open on two sides by means of an opening 12 and an opening 13. The air cushions can be inserted and/or rearranged through these openings. The arrangement of the holder space is such that the cushions are situated round the heel in the position of use by means of channel parts 6 and 7.

Air cushion holder 3 further comprises two channel parts 4, 5 for arranging parts of the air cushions therein. The air cushion holder extends between the respective channel parts 4,6 and 5,7 such that two parallel air cushions can be arranged therein, or that two transverse air cushions can be arranged therein.

The arrangement of air cushions 25, 26 has the advantage that the Achilles tendon can rest between the two air cushions and that it can hereby be supported in freely suspended and unloaded manner by adjacent parts of the ankle or the leg. The air cushion parts in channel parts 4,5 provide lateral support or provide a support without load when the leg is rotated and is held in a lateral orientation.

In assembled form the sides of the device used on the underside of the foot are mutually attached along stitched seam 11. The space 3 for the air cushions is defined by stitched seams 18 on the outer side thereof and stitched seams 16 on the inner side thereof. In the assembled state the openings 12, 13 remain open, which means that only one of the two layers which form space 3 at that position are stitched together along stitched seam 11.

In an alternative embodiment 41 the air cushions are arranged transversely in space 3. That is, air cushions 55 extend in an arc through channel parts 6 and 7 and air cushion 56 extends substantially transversely in channel parts 4,5.

In a further preferred embodiment the part extending around the front foot is already given a closed form during production. An elastic part is for instance applied here along the top side of the foot so that this part can be tensioned under bias round the foot, and the foot hereby remains fixed. A velcro tape is in this embodiment provided on the side along the lower leg and/or above the ankle in similar manner as in the previous embodiments.

This embodiment also has a further air cushion which is arranged at the position of the bulge of the heel. A further isolation of the heel is for instance provided by means of this embodiment, and it is further impossible in this embodiment for the heel to protrude through the opening if the space created between the equivalents of air cushions 25, 26 create insufficient space in this embodiment to hold the heel suspended.

By applying the not fully filled air cushion it becomes possible for the load to be spread over a large surface area of the heel, since this air cushion is able to form itself round the heel. The remaining load is hereby spread in gentle manner over a maximum skin surface area. The use of the separate air cushion as a separate chamber provides the advantage that there is no overflow of a gas in the direction of the two other elongate air chambers, thereby preventing all air being guided away and the heel still possibly being subjected to direct pressure from the mattress.

A further preferred embodiment (FIG. 5A-C) relates to a heel protector 61 which, just as the above preferred embodiments, is intended to protect the heel. This heel protector comprises a number of chambers 62-67 for holding buffer members 71-79 therein. Each of the buffer members serves an individual purpose for a good protection. In this preferred embodiment the buffer members also comprise an airtight or substantially airtight membrane for holding a gas enclosed therein. It is important here that a gas is chosen which cannot or substantially cannot pass through the membrane.

As in the previous preferred embodiments, heel protection devices 61 comprises an encasing body 2. This encasing body 2 comprises integrated therein the different chambers for holding the buffer members or air cushions 71-79 therein. On the rear side of the encasing body is situated a calf chamber 63 which extends from the lower side of the calf to substantially the upper side of the heel. This calf chamber is enclosed by stitched seams 83 on the sides and stitched seam 82 on the underside. These stitched seams define the outer side of the calf chamber. A further stitched seam 68 for the purpose of separating the left part of calf chamber 63 and the right part of calf chamber 62 extends substantially close to the centre of the calf chamber. Air cushions 71-74 arranged in the calf chamber are hereby better held in the desired orientation. Air cushions 71-72 are situated in the left calf chamber and air cushions 73-74 are situated in the right half of the calf chamber. The air cushions extend over the full length of the calf chamber, so from the top edge to the stitched seam 82 on the underside thereof.

Extending in transverse direction over the calf chamber along this stitched seam 82 is an Achilles chamber 64 for holding a further air cushion 75 therein. As seen from the rear, air cushions 71-74 and air cushion 75 respectively therefore have an overlap. When the wearer of the heel protection device rests with a stretched leg on the mattress, the heel is hereby held largely clear of the underlying mattress on the rear side thereof.

Air cushions 71-74 here provide a tissue-relieving contact with the respective parts of the lower leg against which they are arranged. Air cushion 75 provides the extra elevation of the heel and an additional protection of the Achilles tendon. The Achilles tendon is further spared in that air chambers 72 and 73 are arranged in the longitudinal direction of the Achilles tendon, whereby they can provide for support with a minimal load on the tissue of the Achilles tendon.

Since a person does not always lie on his/her back with a leg outstretched, but also with a bent leg, wherein the heel would make direct contact with the mattress and the so-called ‘digging’ can for instance occur, a heel chamber 65 is provided for holding two heel protection cushions 76, 77 therein. This heel chamber extends between stitched seam 82 and the two stitched seams 86, 87 as far as stitched seam 81, which is arranged in the position of use under the underside of the foot. Both these air chambers are arranged close-fittingly in this heel chamber.

The filling of air chambers 76, 77 is here also such that, when supporting thereon, the load is distributed over the greatest possible surface area of the heel, and that the heel makes no direct contact, or a minimal direct contact, with the mattress. The load on the tissue of the heel is hereby minimized, even when a person lying on his/her back with the leg in a bent orientation supports for a long period on the heel.

Situated on both sides of the heel protection device are two side chamber 66, 67 for holding side protection air cushions 78, 79 therein. These air cushions provide a protection to the side of the foot. These air cushions also provide protection for the protruding ankles. Use is also made here of respective air chambers 71, 74 of the calf chamber and air chamber 75 of the Achilles chamber, which also provide a volume on the side of the foot for sparing the tissue of the ankles.

A further example of wear which occurs is the result of so-called ‘digging’, wherein a person lying on his/her back makes a movement between a stretched leg and a bent leg, wherein the heel makes contact with the sheet and/or the mattress. Such a movement creates a load on the heel which can result in uncomfortable symptoms. This so-called ‘digging’ can occur intentionally or unintentionally and need not be an elongate movement, but can also be an almost stationary ‘wiggling’ of the heel and the lower leg. In the application thereof the heel protector is arranged in the shown manner round the foot and the ankle and the lower leg, or at least parts thereof. The heel protector can here be fastened by means of elongate tongues 22 comprising velcro and velcro strips 21 on the other side of the heel protector.

As in previous embodiments, three velcro fasteners 22 are provided for holding the protection device correctly positioned as a whole. Due to the construction of the embodiment the foot is also held in a substantially 90-degree orientation, as shown specifically in FIG. 5B. This prevents the possible forming of a so-called clubfoot. A clubfoot is in fact a muscle disorder in the lower leg, wherein the calf muscle becomes relatively short and the shin muscle becomes relatively long, whereby the foot is forced into a stretched orientation. After a long period of lying on the back this usually happens under the influence of the weight of blankets. The application of this embodiment therefore prevents a bedridden person starting to suffer from such a condition. The structure of the device makes it possible to prevent this so-called clubfoot even when the velcro fastener is applied loosely.

The present invention is described in the foregoing on the basis of several preferred embodiments. Different aspects of different embodiments are deemed described in combination with each other, wherein all combinations which can be made by a skilled person on the basis of this document must be included. These preferred embodiments are not limitative for the scope of protection of this document. The rights sought are defined in the appended claims.

1. A heel protection device for protecting a heel against damage or irritation, such as decubitus symptoms, comprising:
(a) an encasing body for encasing a part of the lower leg and/or the foot, wherein the encasing body comprises at least a buffer member holder for holding at least one buffer member, and
(b) one or more buffer members which can be arranged in the encasing body for the purpose of supporting the heel and/or parts surrounding the heel:
wherein the encasing body can be deformed for close fitting to the heel,
wherein the buffer members comprise at least a fluid chamber which can be sealed by means of a substantially fluid-tight membrane which is deformable,
wherein a buffer member is arranged transversely relative to and opposite the Achilles tendon, and
wherein two or more buffer members are arranged such that in use they extend on either side in the longitudinal direction of the Achilles tendon,
whereby the protruding part of the heel can be freely suspended while the foot rests on the surrounding part via the buffer members.

2. The heel protection device as claimed in claim 1, wherein at least two buffer members or parts of the buffer members are arranged such that at least a part thereof each supports a side of the ankle or the lower leg when it is resting on a surface such as a mattress.

3. The heel protection device as claimed in claim 1, wherein at least one buffer member or parts of the buffer members is arranged such that at least a part thereof supports the foot when it is resting on one of the sides thereof.

4. The heel protection device as claimed in claim 1, wherein the fluid-tight membrane comprises a substantially wholly or wholly fluid-tight plastic, such as a PE, EVOH, PVDC and the like, preferably with a thickness of 30-100 micron, more preferably 30-80 micron, more preferably 30-70 micron, more preferably 30-60 micron.

5. The heel protection device as claimed in claim 1, wherein the volume of the buffer member is partially filled under regular air pres-sure with a gas, such as 20-100%, 30-90%, 40-90%, 50-90%, 60-90%, 70-90% and the like.

6. The heel protection device as claimed in claim 1, wherein a part of a maximum volume of the buffer member is filled with a liquid, such as 20-100%, 30-90%, 40-90%, 50-90%, 60-90%, 70-90% and the like.

7. (canceled)

8. (canceled)

9. The heel protection device as claimed in claim 1, wherein at least one of the buffer members has an elongate cushion shape.

10. The heel protection device as claimed in claim 1, wherein at least one buffer member, such as the heel support buffer member, has a substantially point-symmetrical shape in one view, such as round, square, rhombic, polygonal and the like, for allowing the heel to rest thereon.

11. The heel protection device as claimed in claim 1, wherein the buffer members can be arranged in a manner such that during use they extend on either side of the foot to a position above the ankle.

12. The heel protection device as claimed in claim 1, wherein the encasing body at least partially comprises a sleeve construction.

13. The heel protection device as claimed in claim 1, wherein the encasing body comprises releasable fastening means such as velcro or laces.

14. The heel protection device as claimed in claim 1, wherein the buffer member holder has a form such that it can hold the buffer member or the buffer members close-fittingly in relation to the adjacent parts of the body.

15. A buffer member as claimed in claim 1, for applying in a heel protection device according to claim 1.

16. (canceled)

17. A method for manufacturing a heel protection device as claimed in claim 1, comprising at least one buffer member according to claim 1, comprising steps for:
(a) manufacturing an encasing body comprising one or more buffer member holders, and
(b) arranging the buffer member or the buffer members in a buffer member holder.
